(19) United States
(12) Patent Application Publication
(10) Pub. No.: US 2025/0057431 A1
(43) Pub. Date: Feb. 20, 2025

(54) Device for Indicating Pressure of Fluids

(71) Applicants: Vikas Raghove, Muncie, IN (US); Punam Raghove, Keene, NH (US)

(72) Inventors: Vikas Raghove, Muncie, IN (US); Punam Raghove, Keene, NH (US)

(21) Appl. No.: 18/235,376

(22) Filed: Aug. 18, 2023

(51) Int. Cl. A61B 5/0215

(52) U.S. Cl. CPC A61B 5/0215; A61B 2505/05; A61M 2025/0001; A61M 2205/3348; A61M 2205/583; A61M 2205/7536

(57) ABSTRACT

The present disclosure relates to a device for indicating pressure of fluids. The device includes a first end to receive the fluid and a pressure indicator having a membrane configured to be deformed in presence of fluid. The membrane is configured to be deformed as per a level of fluid the device, thereby indicating the pressure of the fluid. The device further includes a base plate having an aperture and a dome shaped element. The membrane is configured to be attached to the base plate and the dome shaped element is configured to cover the membrane, thereby preventing spilling of fluid. The dome shaped element is made of transparent material, thereby facilitating visual indication of deformation of the fluid. The device is configured to be used in detecting a type of blood vessel, such as, an artery or a vein.

### FIELD OF THE DISCLOSURE

The present disclosure generally relates to an arterial puncture indicator ability to quickly and easily identify presence of tip of needle in the lumen of an artery vs the vein.

### BACKGROUND

Ability to identify an artery or a vein whenever inserting a needle or catheter is critical in many applications. For example, central venous catheters are employed to administer therapeutic fluids and drugs into the larger blood vessel for example near the neck namely internal jugular, subclavian or near groin namely femoral. They are also used for measuring cardiac pressures and some procedures like insertion of transvenous pacemaker and trans vascular procedures. Traditionally, for inserting Central venous catheter to a patient the location of the vein is identified by a hollow needle advanced through the skin until blood is aspirated. The color of the blood, the rate and pulsatility of its flow determines whether the blood is arterial or venous blood. The Seldinger technique is the most common method for inserting a central intravenous (IV) line or Central venous catheter in the blood vessels. The technique involves puncturing the blood vessel with a needle (generally known as introducer needle), after that a blunt guidewire is threaded through the needle, thereafter the needle is removed and a flexible dilator is threaded over guidewire to dilate the track then dilator is removed and catheter is threaded over the guidewire, and finally the guidewire is removed.

One of the major concerns in intravenous (IV) procedures is the accidental puncture of the artery. Since this accidental puncture of the artery has adverse effects for the patient especially if it is unrecognized and dilator or catheter in used, therefore it becomes imperative for the medical practitioner to be highly skilled and vigilant while performing the cannulation procedure. Conventionally, the syringe is removed from the needle hub to see the blood coming out and if the blood spurts out (due to the high pressure of the arterial blood) it indicates that the artery is punctured. This conventional procedure may lead to air embolism, wherein when the medical practitioner removes the syringe from needle hub there are chances of air getting sucked into the blood vessel, specifically if the introducer needle is inside the vein. This can cause serious complications and in some cases can be fatal for the patient especially when the patient is dehydrated. And during intended insertion of catheter to an artery is being performed to know the continued presence of tip of the needle in the artery is important so that guidewire does not create a false track and do tissue damage.

To eliminate the complications, in surgical procedures, caused due to accidental puncture of unintended blood vessels, different devices and modifications are being made while designing pressure indicator for indicating pressure and pulsatility of the punctured blood vessel.

### SUMMARY OF THE DISCLOSURE

The present disclosure sets forth a device for indicating a pressure range and pulsatility of fluid. The device comprises a first end and a dynamic pressure indicator. The first end comprises a cross-section and being configured to receive the flow of the fluid. The pressure indicator is configured to indicate the pressure of the fluid. The pressure indicator comprises a membrane to indicate a level of the pressure of the fluid. The whole assembly can be separate entity or part of an introducer needle in which first end will be a continuation of the hub of introducer needle

The membrane is configured to have a resting position and is further configured to be deformed from the resting position to a deformed position corresponding to the level of the pressure of the fluid.

The pressure indicator comprises a base plate having a first thickness, the base plate being configured for receiving the membrane there within. The base plate is configured to have a top surface, a bottom surface, and an aperture. The aperture extends from the top surface to the bottom surface, and wherein the membrane is configured to cover the aperture on the top surface.

### BRIEF DESCRIPTION OF THE DRAWINGS

A more complete understanding of the present disclosure may be derived by referring to the detailed description and claims when considered in connection with the Figures, wherein like reference numerals refer to similar elements throughout the Figures.

FIG. 1 illustrates an exemplary isometric view of a device for indicating pressure of fluid in accordance with the present disclosure.

FIG. 2 illustrates an exemplary device showing a base plate of a device in accordance with the present disclosure.

FIGS. 3-4 illustrate exemplary top and side views of a device for indicating pressure of fluid in accordance with the present disclosure.

FIG. 5 illustrates an exemplary exploded view of a device for indicating pressure of fluid in accordance with the present disclosure.

### DETAILED DESCRIPTION

The following description sets forth exemplary embodiments of the invention only, and is not intended to limit the scope, applicability, or configuration of the invention. Rather, the following description is intended to provide a convenient illustration for implementing various embodiments of the invention. As will become apparent, various changes may be made in the function and arrangement of the elements described in these embodiments without departing from the scope of the invention as set forth herein. It should be appreciated that the description herein may be adapted to be employed with alternatively configured devices having different shapes, components, attachment mechanisms, and the like and still fall within the scope of the present invention. Thus, the detailed description herein is presented for purposes of illustration only and not of limitation.

Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

Embodiments of the present disclosure describe a device for dynamically indicating pressure of fluid. The device includes a first end for receiving the flow of the fluid and a pressure indicator for indicating the pressure of the fluid. The pressure indicator includes a membrane for detecting a level of pressure of the fluid. It is to be noted that the membrane is used for exemplary purposes and the level of fluid may be indicated using similar structure such as, a diaphragm and the like. The device is configured to indicate a level of pressure of fluid (i.e., blood) in blood vessels. A material of the membrane can be selected according to the application. For instance, in case where the pressure of the fluid is less, the more flexible membrane is used, and in case of more pressure of the fluid, the membrane with more rigidity can be used.

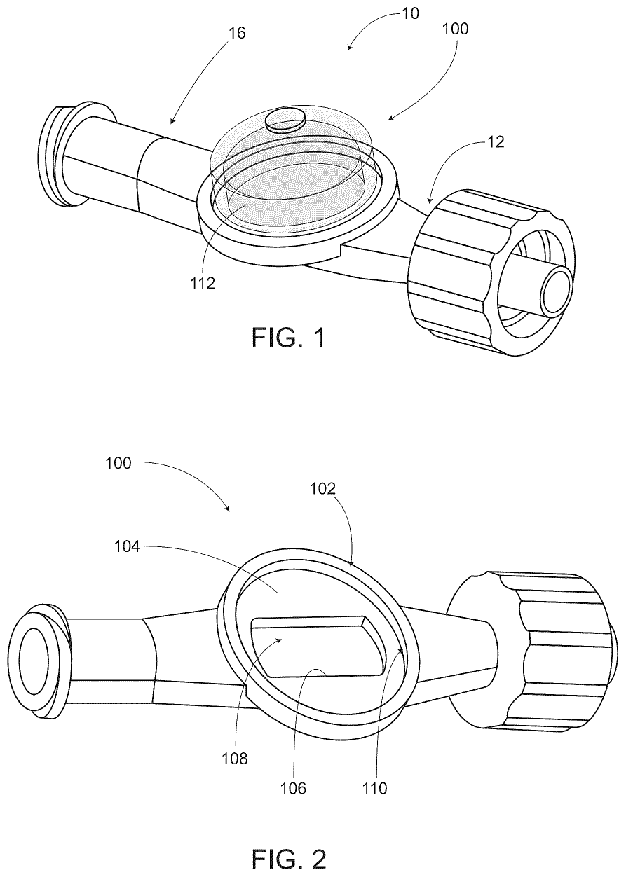

Reference is now made to FIG. 1, which illustrates a device 10 for indicating pressure of fluid in accordance with the present disclosure. The device 10 is defined by a first end 12 and a second end 16. The device 10 is configured to receive a fluid at the first end 12 and is configured to form a fluid path that extends from the first end 12 to the second end 16. The device 10 is configured to be connected to an inlet means (as will be explained hereinbelow) at the first end 12 for receiving the fluid.

The device 10 further comprises a pressure indicator 100 for indicating a level of pressure of the fluid received at the first end 12. It is to be noted that even though it is indicated that the fluid is received at the first end, it is possible to receive the fluid from the second end 16 as well, without departing the scope of the present disclosure. Accordingly, the device is configured to indicate the level of fluid received from the first end 12 as well as from the second end 16.

The pressure indicator 100 comprises a membrane 112 for indicating the level of pressure of the fluid. The membrane 112 is configured to have a resting position when there is no flow of the fluid. Accordingly, the membrane 112 is at the resting position in an inoperative stage. The membrane 112 is configured to be deformed in presence of the fluid in the device 10. The membrane 112 is configured to be deformed from the resting position to a plurality of deformed positions based on the level of the fluid. In an embodiment, in presence of a first pressure of the fluid, the membrane 112 is configured to be deformed from the resting position to a first deformed position. Similarly, in presence of a second pressure of the fluid, the membrane 112 is configured to be deformed from the resting position to a second deformed position. The first pressure may be less than or more than the second pressure. Accordingly, the first deformed position is different than the second deformed position based on the first level and the second level of pressure of the fluid. Accordingly, the deformation of the membrane in the first deformed position is distinguishable from the second deformed position, and hence, the deformation and the level of fluid is easily visually differentiable. In an exemplary embodiment, the first level of pressure is 4 mmHG and the second level of pressure is 60 mmHG. Accordingly, the membrane 112 deforms to the first deformed position and to the second deformed position from the resting position. It is to be noted that there is a significant difference in the pressure of the fluid at the first level of pressure and the second level of pressure. Hence, the deformation of the membrane 112 is visible by a user of the device 10. Accordingly, the device 10 facilitates easy indication of the level of the pressure of the fluid. In some embodiments, the device 10 may be used to indicate a first pipe having a high level of pressure of the fluid and a second pipe having low level of pressure of the fluid. For instance, the upon use, if the level of pressure is high, then it can be inferred that the pipe is the first pipe having fluid with high pressure. Similarly, if the pressure is low, then it can be inferred that the pipe is the second pipe having fluid with low pressure.

Additionally, the membrane 112 is configured to be deformed after a predefined amount of pressure of the fluid. In other words, the membrane 112 does not deform to the first deformed position or the second deformed position until the predefined pressure of the fluid is reached. The material of the membrane 112 is selected such that the membrane 112 does not deform in pressure of a small change in the pressure of the fluid. In an exemplary embodiment, the membrane 112 is configured not to be deformed if the pressure of the fluid is around 0.25 to 0.50 mmHg. Hence, in case of a very small change is the pressure of the fluid, the membrane 112 does not deform, thereby eliminating incorrect indication of the pressure of the fluid.

It is to be noted that in a material for the membrane 112 may be selected so as to make the membrane 112 flexible and elastic to clearly provide visual difference in deformation of the membrane 112 according to the level of pressure of the fluid. Moreover, the membrane 112 is selected to be a fluid-proof membrane 112. In other words, the membrane 112 does not allow transfer of fluid from one side to the other side therethrough, thereby facilitating in indication of an actual pressure of the fluid. In an exemplary embodiment, the membrane 112 may be made of platinum-cure silicone and may have a circular shape having diameter of 1 cm. The membrane 112 may have a thickness of 200 μm and have hardness of 5 durometers.

In some exemplary embodiments, the membrane 112 may comprise a restraining element attached at an upper surface and/or a lower surface thereof. The lower surface is defined as the surface facing the fluid passing through the device 10. In other words, the lower surface of the membrane 112 is the surface over which the pressure of the fluid is applied, that results in deformation of the membrane 112. The upper surface is positioned at the opposite side of the lower surface and generally faces towards the outer side of the device 10. The restraining element prevents release of the membrane 112 until the pressure of the fluid exceeds a predetermined threshold value.

In some embodiments, the pressure indicator 100 may include a base plate 102 between the first end 12 and the second end 16. The base plate 102 may serve as a means to support the membrane 112. FIG. 2 illustrates an exemplary base plate 102 in accordance with the present disclosure. The base plate 102 is configured to have a top surface 104 and a bottom surface 106. The membrane 112 may be placed within the base plate 102 and attached to the top surface 104 thereof. In an embodiment, the dimension of the membrane 112 is selected such that the membrane 112 is comfortably received in the base plate 102. In an exemplary embodiment, the membrane 112 may be attached to the top surface 104 of the base plate 102 using a thin layer of a sealant room temperature vulcanizing (RTV) silicone around its circumference over the top surface 104. This sealant has a reasonable open time and allows exact positioning of the membrane 112. Alternatively, a crosswise thin bridge may be used to hold a low-strength adhesive to keep the membrane 112 from rising to indicate pressure until pressure exceeds a small amount to prevent a very minimal pressures from causing a visible false deformation in the membrane 112. The false deformation may lead to erroneous indication of pressure of the fluid.

The base plate 102 further includes an aperture 108 that extends from the top surface 104 to the bottom surface 106. The aperture 108 is positioned such that the flow passes below the bottom surface 106 of the base plate 102, and hence, of the aperture 108. In other words, the aperture 108 is positioned in the fluid path. When the membrane 112 is attached to the top surface 104 of the base plate 102, the membrane 112 is also positioned above the aperture 108. In other words, when the fluid is received in the first end 12 of the device 10, the aperture 108 provides fluid communication between the fluid and the membrane 112, thereby allowing the membrane 112 to be deformed in presence of the fluid and facilitating indication of the pressure of the fluid.

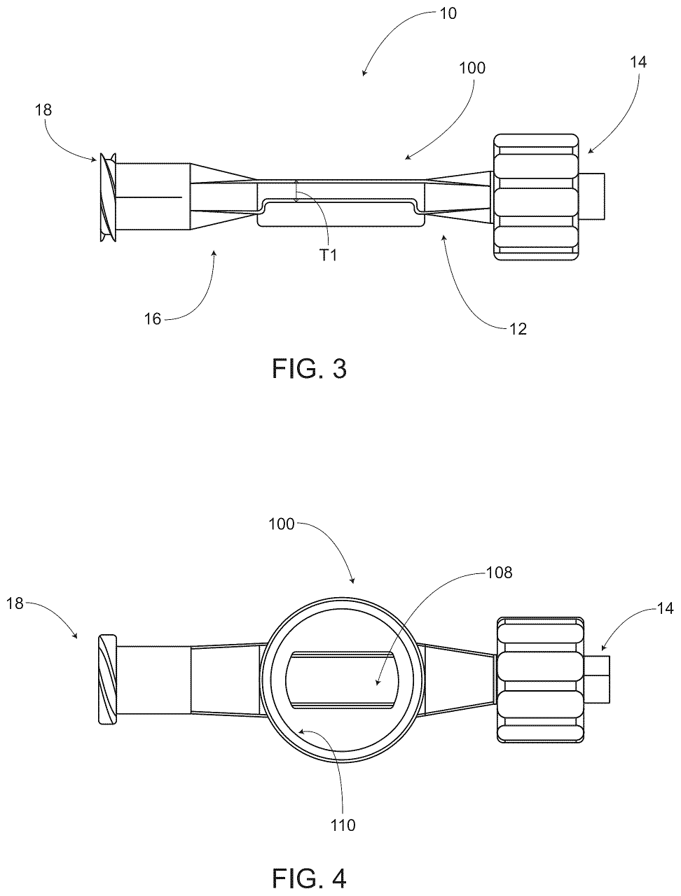

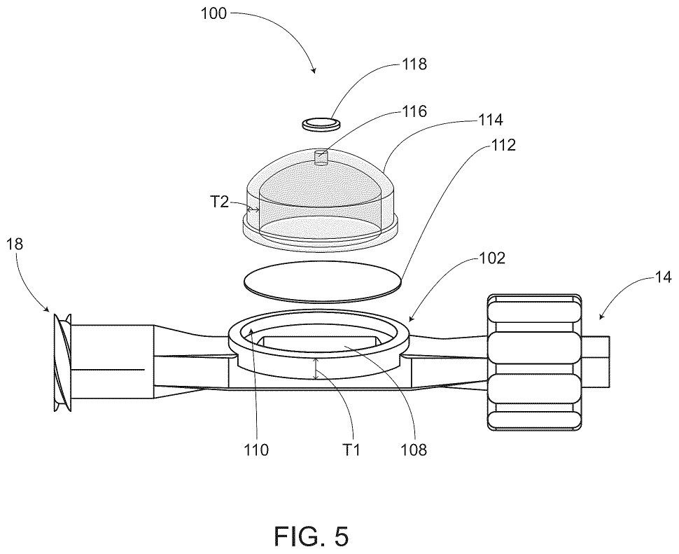

FIGS. 3 and 4 illustrate exemplary side and top views of the device 10 having the base plate 102. The base plate 102 is placed between the first end 12 and the second end 16 of the device 10 and is configured to have a first thickness T1. The first thickness T1 is selected so as to comfortably receive the membrane 112 therewithin. In other words, the first thickness of the base plate 102 is more than the thickness of the membrane 112 such that the membrane 112 is securely attached to the top surface 104 of the base plate 102. The base plate 102 further includes an annular ring 110 having the first thickness T1. The annular ring 110 is also configured to provide secure attachment to the membrane 112. The annular ring 110 may have a predefined height and may generally follow periphery of the base plate 102. The predefined height facilitates support and secure attachment of a dome shaped element 114 (as will be described hereinbelow with respect to FIG. 5) with the base plate 102.

It is to be noted that even though the base plate 102 has been discussed here for supporting the membrane 112, such base plate 102 does not limit the scope of the invention and any alternative means or suitable modification are included well within the scope of the invention.

Reference is now made to FIG. 5, which illustrates an exemplary exploded view of the device 10 having the pressure indicator 100. The pressure indicator 100 includes a dome shaped element 114 configured to cover the membrane 112. The dome shaped element 114 is configured to be made of a transparent material so that the deformation of the membrane 112 is clearly visible. The dome shaped element is further defined by a wall that is of cylindrical shape so as not to introduce optical distortion which may impair visibility of the membrane. In addition, the shape of the dome shaped element 114 is designed so that a dome of the element comfortably receives/houses the membrane 112 in its fully deformed state. In other words, the dome shaped element 114 is configured to house the membrane 112 when the level of pressure of the fluid is high such that the membrane 112 has deformed forming almost a semi-sphere.

The dome shaped element 114 comprises a second thickness T2 and is configured to be attached to the base plate 102. The dome shaped element 114 may be received within the annular ring 110 arranged in a periphery of the base plate 102. Accordingly, the dome shaped element 114 is configured to be placed over the membrane 112 on the top surface 104 of the base plate 102. The dome shaped element 114 may be clamped to the annular ring 110 of the base plate 102 and may be held thereon using an adhesive. The second thickness T2 is configured to facilitate secure attachment of the dome shaped element 114 with the base plate 102. One non-limiting example of the adhesive is wicking-grade cyanoacrylate adhesive.

The dome shaped element 114 includes a vent hole 116. The vent hole 116 is placed at the farthest part of the dome shaped element 114 from the base plate 102. In an exemplary embodiment, the farthest part is referred as a top of the dome the dome shaped element 114. The vent hole 116 is configured to be sealed by a hydrophobic filter 118. Accordingly, there is created a first volume space that is between the membrane 112 and the vent hole 116. As the filter 118 is a hydrophobic filter, there is fluid communication between the first volume space and the surrounding atmosphere. Accordingly, at the time of deformation of the membrane 112, a first volume of the air from the first volume space is displaced to the surrounding atmosphere. Similarly, when the membrane 112 is displaced to its resting position, a second volume of air from the surrounding atmosphere is received within the first volume space.

The filter 118 further restricts passing of liquid from the first volume space to the surrounding atmosphere and vice versa. This feature prevents spilling of the liquid from the device 10 to the surrounding atmosphere. Specifically, accidental rupture of the membrane 112 or detachment of the membrane 112 from the top surface 104 of the base plate 102 can spill the fluid into its surrounding. The rupture of the membrane 112 provides a second fluid path to the fluid in the device 10. Hence, the fluid flowing through the first end 12 goes to the dome because of the accidental rupture of the membrane 112. Because of the hydrophobic filter 118, even if the membrane 112 is ruptured, the fluid is not spilled outside the device 10. Hence, the filter 118 works as a spillage prevention mechanism in the device 10. It is to be noted that the filter 118 is used for mere exemplary purpose and does not limit the scope of the invention. Any suitable means to restrict flow of the fluid can be used as spillage prevention mechanism.

The device 10 may be configured to be attached to an inlet means 14 at the first end 12. The inlet means 14 may be configured to receive the flow of the fluid. In an exemplary embodiment, the device 10 may be used to detect puncture of an artery, or to determine whether the punctured blood vessel is an artery or a vein. The pressure of blood in the artery is comparatively higher than the pressure of blood in the veins. Based on the level of pressure, the membrane 112 deformed to the first deformed position or to the second deformed position. A practitioner can readily determine by deformation of the membrane 112 visually the type of blood vessel punctured. Accordingly, the practitioner may proceed with the further steps. For instance, if an artery is accidentally punctured, corresponding preventive steps may be taken by the practitioner. In such embodiment, the first end 12 of the device 10 is configured to be connected with an introducer needle (not shown). The introducer needle acts as an inlet means 14 in such embodiment. The introducer needle is used to puncture the blood vessel. Upon puncturing, the blood of that blood vessel is received within the device 10. Based on the pressure of the blood in that blood vessel, the membrane 112 is deformed, thereby visually indicating the type of blood vessel punctured. It is to be noted that the device 10 may be externally attached to the introducer needle or may be integral part of the introducer needle. Some other non-limiting examples of the inlet means 14 are standard hypodermic needles, catheters, tubing with Luer connectors, and the like. It is to be noted that in such embodiments, the device 10 is not directly connected to the blood vessels, and is configured to receive fluid (i.e., blood) using the inlet means 14.

In some embodiments, the second end 16 of the device 10 may not be connected to an outlet means 18. Alternatively, the device 10 may be connected to an outlet means 18 at the second end 16. Some non-limiting examples of the outlet means 18 are a syringe, an intravenous (IV) bag, and the like. In the embodiments where an outlet means 18 is connected at the second end 16, the fluid path is established from the inlet means 14 to the outlet means 18.

The device 10 is configured to provide a visual indication of a level of the fluid. Hence, the device 10 is easy to use. Also, the device 10 is simple and low-cost, because of the use of a smaller number of components.

Finally, while the present invention has been described above with reference to various exemplary embodiments, many changes, combinations, and modifications may be made to the exemplary embodiments without departing from the scope of the present invention. For example, the various components may be implemented in alternative ways. These alternatives can be suitably selected depending upon the particular application or in consideration of any number of factors associated with the operation of the device. In addition, the techniques described herein may be extended or modified for use with other types of devices. These and other changes or modifications are intended to be included within the scope of the present invention.

What is claimed is:

1. A device for indicating a pressure and pulsatility of a fluid, the device comprising:
a) a first end having a cross-section and being configured to receive a flow of the fluid; and
b) a pressure indicator configured to indicate the pressure of the flow;
wherein the pressure indicator comprises a membrane configured to indicate a level of the pressure of the flow.

2. The device of claim 1, wherein the membrane is configured to have a resting position and is further configured to be deformed from the resting position to a deformed position corresponding to the level of the pressure of the fluid.

3. The device of claim 2, wherein the membrane is configured to be deformed from the resting position to a plurality of deformed positions.

4. The device of claim 3, wherein the membrane is configured to be deformed from the resting position to a first deformed position on detection of a first pressure of the fluid, thereby indicating a first level of pressure of the fluid, and is configured to be deformed to a second deformed position on detection of a second pressure of the fluid, thereby indicating a second level of pressure of the fluid.

5. The device of claim 1, wherein the pressure indicator comprises a base plate having a first thickness, the base plate being configured for receiving the membrane therewithin.

6. The device of claim 5, wherein the base plate is configured to have a top surface, a bottom surface, and an aperture, wherein the aperture extends from the top surface to the bottom surface, and wherein the membrane is configured to cover the aperture on the top surface.

7. The device of claim 5, wherein the base plate comprises a top surface having an annular ring, the annular ring being configured to receive a dome shaped element.

8. The device of claim 7, wherein the dome shaped element is made of a transparent material and is configured to cover the membrane.

9. The device of claim 7, wherein the dome shaped element comprises a vent hole configured to be covered by a filter.

10. The device of claim 9, wherein the filter is a hydrophobic filter.

11. The device of claim 1, wherein the membrane is configured to have a membrane thickness for facilitating displacement thereof in presence of the fluid.

12. The device of claim 1, wherein the membrane is configured to have a restraining element attached at an upper surface and/or a lower surface thereof, the restricting element being configured to allow movement of the membrane in presence of the fluid having a pressure at least equal to a threshold value.

13. The device of claim 1, wherein the first end is configured to be attached to an inlet means at the first end, the inlet means configured to receive the fluid.

14. The device of claim 13, comprising an outlet means attached to a second end of the device, the outlet means configured to receive the fluid from the inlet means through the device and to generate a fluid path.

15. The device of claim 1, wherein the device is configured to indicate pressure of blood in blood vessels.
